(19) United States
(12) Patent Application Publication
(10) Pub. No.: US 2020/0345628 A1
(43) Pub. Date: Nov. 5, 2020

(54) Novel Route of Nasal Administration of Aripiprazole for Treatment of Eating Disorders

(71) Applicant: Alex John Shepherd, Rochester, MN (US)

(72) Inventor: Alex John Shepherd, Rochester, MN (US)

(21) Appl. No.: 16/402,231

(22) Filed: May 3, 2019

(51) Int. Cl. A61K 9/00; A61M 15/08

(52) U.S. Cl. CPC A61K 9/0043; A61B 5/4866; A61K 9/0073; A61M 15/08

(57) ABSTRACT

The use of a novel route of administration of aripiprazole for treatment of an eating disorder. Specifically, the use of an intranasal route of administration of aripiprazole for treatment of an eating disorder such as anorexia nervosa, atypical anorexia nervosa, bulimia nervosa, binge-eating disorder, purging disorder, or any combination of multiple types of eating disorders, as well as any syndrome disease such as night eating syndrome, carcinoid syndrome, or serotonin syndrome or any diseases or symptoms comorbid with an eating disorder. This intranasal route of administration will most likely reach the brain via the single epithelial cell layers of the nose to blood vessels to the cerebrospinal fluid neighboring the olfactory bulbs and finally into the nervous system where it may produce both local or systemic effects. This route may treat neurotransmitter imbalances, altered eating behaviors, or comorbid symptoms or diseases associated with an eating disorder including treatment and compliance.

### RELATED APPLICATIONS

This application is a continuation-in-part of U.S. provisional patent 62809623 filed Feb. 23, 2019.

### FIELD

This is in the field of pharmacology. The present invention is for a pharmacological treatment method for eating disorders. This invention is for the use of a nasal route of administration of aripiprazole for treatment of eating disorders, such as the administration of aripiprazole via a syringe with attached intranasal mucosal atomization (MAD) device, a pump spray bottle, depot injector, pre-filled syringe, auto-injector, needleless syringe, chambered syringe, dual chambered syringe, or other type of nasal dispenser unit or injection device. This novel nasal route of administration of aripiprazole will primarily be used for anorexia nervosa but may also include other eating disorders such as but not limited to atypical anorexia nervosa, bulimia nervosa, binge-eating disorder, purging disorder, or any combination of multiple types of eating disorders, syndromes such as night eating syndrome, carcinoid syndrome, or serotonin syndrome, or any diseases or symptoms comorbid with an eating disorder.

### Background

Eating disorders have the highest mortality rate of any psychiatric disorder (Birmingham et al., 2005). This high mortality rate is probably a result from current treatment methods being ineffective. Understanding why current treatment methods do not work is imperative to creating more effective solutions for treating eating disorders.

An eating disorder can produce a wide array of symptoms and thus most likely stems from an emergent combination of many different biopsychosocial factors. Additionally, eating disorder symptoms may vary slightly by disorder. One eating disorder I will focus on in the background is anorexia nervosa (AN). This disease is classically characterized by the constant desire to lose weight, (Heaner & Walsh, 2013). However, the disorder also includes psychiatric factors such as the fear of gaining weight, distorted body image, and emotional abnormalities (Watson & Bulik, 2013).

My theory is that an eating disorder, specifically AN, is partially caused by a biological predisposition to excessive serotonin neurotransmitter levels and reduced dopamine neurotransmitter levels in the brain. This imbalance of serotonin and dopamine ultimately leads to increased amygdala activation and emotional abnormalities such as depression, anxiety, and negative affect. These emotional abnormalities such as negative affect and altered emotional processing in turn leads to starvation as a compensatory response to improve mood. As such, understanding negative affect in those with AN is imperative toward finding an effective treatment method. However, due to the body dysmorphia and anxiety of weight gain experienced in those with AN, those with AN may not want to take just any treatment that increases weight gain without also addressing their neurotransmitter imbalances and emotional symptoms experienced in AN as well. As such, finding a treatment that not only works but is tolerated is imperative for curing AN.

To help prove this theory described above, a top-down approach is needed. First, it seems that emotion plays a role in disordered eating behavior. This is supported by Engel and colleagues (2013) who show in the paper that negative affect in those with AN is associated with more severe eating disorder symptoms. In other words, they showed that individuals with AN were restricted their caloric intake more when their moods were more negative (Engel et al. 2013b). Interestingly, they also found that negative mood increased after they lost control over the calories they consumed or when weighing themselves. Thus, understanding emotion and incorporating this understanding into new treatment methods is imperative toward a cure for AN.

Moreover, emotional abnormalities are directly present in AN. For example, those with AN demonstrate higher levels of negative emotion compared to healthy counterparts (Oldershaw et al., 2015). AN is also associated with depression and anxiety (Galimberti et al., 2013; Halmi et al., 2012; & Wild et al., 2016). These psychiatric factors such as depression and anxiety can also impact the treatment outcomes of anorexia (Wild et al., 2016 & Bloss et al., 2011). For example, childhood anxiety before AN onset is predictive of worse treatment outcome and increased AN complications such as more severe weight loss (Bloss et al., 2011). Similarly, lifetime depression is negatively associated with BMI and AN recovery (Wild et al., 2016).

If negative emotion is playing a role in anorexia nervosa, then an important question becomes what is causing this negative emotion to alter eating behavior? To do this, one must consider an area of the brain important for negative emotion called the amygdala. Interestingly, research has shown that amygdala activity is elevated in those with AN, as demonstrated by a functional magnetic resonance imaging (fMRI) study showing elevated blood oxygen level dependent responses of the left amygdala in individuals with AN when shown photos of high caloric drinks (Ellinson et al., 1998 & Joos et al., 2017). Those with AN also show increased amygdala activity compared to controls when seeing food, when thinking about eating, but also when physically ingesting food (Vocks et al., 2011). The amygdala is strongly associated with negative affect meaning mood is greatly reduced when amygdala activity is high (Barrett et al., 2007). Moreover, reducing the activity of the amygdala reduces negative affect (Silvers et al., 2012). As such, if altered eating behavior is worsened by greater negative affect and if greater negative affect is occurring as a result of increased amygdala activity, then finding what is causing this increased amygdala activity should help reduce altered eating behavior in those with AN. To do this, finding what is causing the amygdala to be excessively activated is needed. As the amygdala is activated by serotonin (Bocchio, McHugh, Bannerman, Sharp, & Capogna, 2016), then perhaps excessive serotonin present in individuals creates an predisposition for hypersensitive amygdala activity, making it easier for those with AN to experience amygdala activation and negative affect. If this were true, then one should see increased baseline serotonin in those with AN compared to healthy controls. Interestingly, researchers have found that serotonin is significantly increased in those with AN (Bergen et al., 2003; Kaye et al., 2005; & Kaye et al., 1991). Individuals with AN also show altered serotonin receptor binding (Kaye et al., 1991), including significantly lower serotonin 5HT2A receptor binding potentials in the frontal cortex (Audenaert et al., 2003). This decreased serotonin receptor binding implies excessive serotonin levels as receptor binding of a ligand will decrease to compensate for the increased ligand present in the brain, in this case serotonin (Audenaert et al., 2003). As such, if the theory is correct, then those with an eating disorder could be not eating as a compensatory way to improve mood. By reducing food intake, this reduces serotonin levels, and thus reduce the amount of serotonin available to activate the amygdala, thus decreasing negative affect. If so, then serotonin levels should directly correlate with eating disorder symptoms. Again, this seems to be the case as reductions in serotonin binding potentials negatively correlated with AN symptoms, specifically drive for thinness (Frank et al., 2002).

Additionally, alterations in serotonin levels directly correlate with the personality traits commonly seen in AN such as harm avoidance, anxiety, obsessionality, and depression symptoms (Kaye et al., 2005). Moreover, it would make sense that if this serotonin imbalance is not corrected, this negative affect bias would continue to occur and may explain why many individuals continue to show increased negative affect even after weight recovery (Oldershaw et al., 2012).

However, this may not be the only cause. AN may also be occurring from alterations in dopamine levels. First, dopamine in the amygdala have been show to regulate limbic processing of aversive factors in humans (Kienast et al., 2008). More specifically, Kienast showed that dopamine storage capacity helps modulate emotional processing in the amygdala and this dopamine relationship is inversely related to trait anxiety (Kienast et al., 2008). Secondly, lesions through dopamine mediated ventral tegmental area are linked to depressive like behaviors in rats (Frisch et al., 2001). As such, taking a top down approach again, if those with AN were low in dopamine, it could be an explanation for the altered emotional processing of the amygdala present in AN individuals.

If this were true, then we should see low dopamine levels in those with AN. Interestingly, low levels of dopamine are present in those with AN (Phillipou, Rossell , & Castle, 2014 & Frank, 2014). The mesocortical pathway in the brain excites the frontal lobe (Blonder & Slevin, 2011). Moreover, the mesocortical pathway is activated by dopamine (Frodl et al., 2002). This need for dopamine to activate these pathways has been shown in both in rats (Oades & Halliday, 1987) and primates (Williams & Goldman-Rakic, 1998). Therefore, dopamine alterations may be an important treatment target for anorexia nervosa.

If mood is affecting eating behavior in those with AN through altered neurotransmitter levels, then a pharmaceutical approach toward normalizing these neurotransmitter imbalances may help. Clinicians initially tried to treat AN with selective serotonin reuptake inhibitors, which have shown some success in depression. However, they showed no significant benefit in helping those with AN and disordered eating behavior symptoms did not change (Ferguson, La Via, Crossan, & Kaye, 1999). This makes sense though as those with AN already have too much serotonin so increasing serotonin through an SSRI will not help their eating disorder symptoms.

Next, some clinicians thought giving an antipsychotic medication would improve AN by reducing altered body image processing. However, results were not just ineffective, but were actually shown to make symptoms worse (Lebow, Sim, Erwin, & Murad, 2013). Specifically, antipsychotics, compared to placebo, did not increase BMD, drive for thinness, or body dissatisfaction and instead increased anxiety and overall eating disorder symptoms (Lebow et al., 2013). Again, this finding fits wells with our model, given that antipsychotics are dopamine antagonists (Lebow et al., 2013) and reducing dopamine in a brain that already has insufficient levels of dopamine would theoretically worsen symptoms.

As such, I believe that the ultimate cure for anorexia nervosa would be a pharmaceutical approach that addresses both the serotonin and the dopamine alterations. More specifically, a serotonin partial antagonist (as serotonin is too high in those with AN) and a dopamine partial agonist (as dopamine is too low in those with AN) would make the most sense.

With this in mind, aripiprazole is both a serotonin partial antagonist and a dopamine partial agonist and would help accomplish these treatment targets by reducing these serotonin and dopamine imbalances and therefore help reduce the altered eating behavior and other eating disorder symptoms experienced from this imbalance. Therefore, I propose the use of the medication, aripiprazole for treatment of AN. This medication should help normalize serotonin and dopamine neurotransmitters and receptor activity in turn improving emotional processing and affect which will subsequently reduce altered eating behaviors and symptoms experienced by those with AN.

However, I believe curing this imbalance and subsequent anxiety and negative affect seen in those with AN is not as simple as giving an medication to cure the disorder. AN has historically been very difficult to treat and tolerance to treatment is a major factor in this difficulty. This extreme difficulty in providing treatment to those with AN is supported by Katherine Halmi who analyzed randomized trials for treatment of anorexia nervosa and found that most studies had very high dropout rates (Halmi, 2008). For example, Bissada et al. 2008, as cited by Halmi, 2008, found that in a treatment trial, 45 perfect refused day hospital treatment and of the ones that agreed, 55 percent declined pharmaceutical treatment (Bissada et al. 2008; Halmi, 2008). That means 75 percent of the patients having anorexia nervosa were not willing to try any pharmaceutical treatment. Additionally, of those willing to try medication, many times adherence is low. For example, in one year long study, treatment adherence was just 56% of those assigned to just oral medication treatment alone (Halmi et al., 2005). In other words, if treatment refusal rate and compliance remains high, those with AN will not get the medication they need to cure the disorder. Finding a way to reduce treatment refusal and increase compliance is paramount for treating AN as without treatment this neurotransmitter balance would remain altered leading to sustained negative affect and altered eating behaviors.

Why this treatment difficulty is so high is not fully understood. I believe treatment crees high psychological distress in those with AN from associations between treatment and weight gain. If if treatment methods increase weight gain without accounting for other aspects of the disorder such as the anxiety toward weight gain and negative affect experienced when eating, it could create psychological distress in those with AN and make those with AN not want to pursue treatment. In other words, altered body image processing seen in those with AN gives them the illusion that they are actually overweight when they are in fact underweight. Thus in their minds, they do not want to seek treatment because they feel doing so would go against their goals of losing weight since they perceive themselves to be “overweight”. As such, this could result in the high treatment refusal rates or compliance issues if the treatment increases their weight without correcting the anxiety or emotional aspect of the disorder. Correcting this neurotransmitter imbalance may help to not just increase weight gain but also help correct the anxiety and emotional aspects of eating as well. However, correcting this neurotransmitter imbalance may not be enough to overcome treatment refusal. I think the way the medication is administered may also make a huge difference.

In other words, I believe that treatment using an oral route of administration is contributing to the extreme difficulty in providing treatment for individuals with AN. This is because those with anorexia become fearful and anxious when ingesting anything orally. As previously discussed above, amygdala activity is elevated in those with AN, as demonstrated by a functional magnetic resonance imaging (fMRI) study showing elevated blood oxygen level dependent responses of the left amygdala in individuals with AN when shown photos of high caloric drinks (Ellinson, 1998 & Joos, 2011). Those with AN also show increased amygdala activity compared to controls when seeing food, when thinking about eating, but also when physically ingesting food (Vocks, 2011). As such, it seems those with AN show increased amygdala activity compared to controls not just when seeing food but also when physically ingesting something as well. In other words, any stimuli taken orally may be producing anxiety and negative affect in those with AN due to a generalized association between oral intake and amygdala activation. As such, attending to any food-related stimuli or having to take anything orally such as a medication could induce fear in those with AN, in turn promoting stress and treatment refusal. Therefore, it seems very possible that the very act of taking medication orally could be a major cause of treatment refusal and or reduced adherence to the medication. If the medication is not taken orally, those with AN may be more inclined to try the treatment method as this would reduce anxiety by bypassing this oral phobia and therefore decrease treatment refusal. As such, finding a way to avoid this oral route of administration may be beneficial.

Oral route of administration may also contribute toward medication refusal due to learned association effects. Humans learn associations between what they eat and its effects on our body due to something called conditioned taste aversion (Lin, Arthurs, & Reilly, 2017). This is to ensure poisonous things are not ingested again. As part of this conditioned taste aversion, humans associate the strongest stimulus with their effects, such as nausea or even anxiety (Lin, Arthurs, & Reilly, 2017). As such, its possible those with AN could wrongly associate the oral medication as the cause of the unwanted side effects such as nausea or weight gain. Moreover, one only has to try something once for this conditioned learning to occur. As such, taking the medication in a different way than orally may help increase willingness to try the medication as well as compliance after starting the treatment.

Third, as those with AN become adjusted to low caloric intake, when treatment does occur and caloric intake increases, their bodies may not be used to the increase in calories and can result in something called refeeding syndrome. Symptoms include weakness, mental confusion, edema, pain in the upper abdomen, dysphagia, nausea, bloating, and constipation (Malczyk & Oswiecimska, 2017). These can be extremely serious and provide both mental, physical, and psychiatric stress to those with AN, especially as those with AN have a severe fear of overweight body image. Therefore, these complications may not only make those with AN not want to eat anything or take anything oral when experiencing nausea or stomach pain, but additionally, it is possible that those taking oral medication may be more likely to associate their refeeding complications from the medication if taken orally instead of the refeeding complications being the problem.

Fourth, those with AN can experience hepatocyte injury and increased liver failure due to malnutrition. This can include hepatitis as well as acute liver failure from coagulopathy and encephalopathy as well as increased liver enzymes as part of the refeeding process due to hepatic steatosis (Rosen et al. 2017). As such, oral medications may provide issues in those with AN as oral route of administration must pass through the hepatic system. Nasal route of administration eliminates this because medication does not have to go to the hepatic system before absorption.

Fifth, other non-oral routes of administration such as intramuscular or intravenous may be painful for those with AN especially if individuals would need to take the dose daily. Therefore finding a non-oral route of administration that does not induce pain would be beneficial.

Sixth, if the medication aripiprazole is expensive, then nasal route of administration may help reduce this cost by reducing the amount of active drug needed for therapeutic dosage as nasal usually requires less overall dosage than oral to be therapeutically effective. This is because the stomach destroys a significant amount of the drug due to stomach acid present via the oral route of administration. Nasal may also be beneficial as it may be faster acting and create a greater active response.

### SUMMARY OF THE INVENTION

The present invention is for the use of a novel route of administration of aripiprazole for treatment of eating disorders, specifically anorexia nervosa (AN) in humans. This route of administration is a nasal route of administration. It may occur via a pump spray bottle device or via a syringe with attached intranasal mucosal atomization (MAD) device. It may also occur via a nasal spray, nasal drops delivering device, nasal douche, or other type of dispenser unit used for the transfer of aripiprazole into the nasal passage or passages for treatment of eating disorders. This route may also contain a pressurized or unpressurized canister and or a space capable of holding the drug or base needed to deliver the compound to the intended area. This route of administration may be delivered primarily through the nasal cavity intranasally but may also have its effect in part via delivery of the compound or compounds to the lungs, skin, muscle, or conjunctiva. It may be delivered into or around the nasal cavity into one nostril or two. This novel nasal route of administration ideally administers a therapeutic dose of aripiprazole into the lining of the nasal cavity which then absorbs into the central nervous system via blood vessels around the mucosal lining. However, this route of administration may also include any transfer of aripiprazole to the olfactory system and may be delivered to and act on any of the following areas: outer and intranasal cavity (both upper and lower nasal cavity), olfactory mucosa, olfactory epithelium, olfactory cells, cribriform plate, cranial cavity, blood brain barrier, brain tissue and cerebrospinal fluid, intracellular, extracellular, cytoplasm, central nervous system or peripheral nervous system. This may also include transfer of aripiprazole passive or actively, naturally or artificially such as via cellular or molecular transport or diffusion, pressure, electrically, magnetically, or mechanically driven into the nasal cavity or cavities.

This novel nasal route of administration is paramount for the hopes of reducing high treatment refusal rates and improving medication tolerance by bypassing oral routes of administration. By bypassing this route, it may help to reduce anxiety from oral phobias, improve treatment experience, and quality of life for those with AN. The use of a specific medication, in this case aripiprazole, is important because it serves as a partial dopamine agonist and partial serotonin antagonist used to correct the neurotransmitter imbalance seen in those with an eating disorder. This novel route may be especially important for eating disorder individuals who cannot or will not take medication orally. It may also help improve compliance by reducing learned associations between the oral medication and weight gain. Third, this route of administration may help improve compliance in those with AN complicated by refeeding symptoms. Fourth, this novel nasal route of administration may help individuals who as a result of the eating disorder suffer from liver problems as nasal route of administration can bypass the hepatic system. Fifth, it may reduce physical pain as this nasal route of administration could be an alternative way to take the medication non-orally without being more invasive such as a painful intradermal needle injection or an intravenous needle injection both of which would puncture the skin and cause pain. Sixth, it may produce a more cost effective and or rapid method of delivering a therapeutic dose.

### DETAILED DESCRIPTION OF THE INVENTION

### Definitions

Anorexia is the loss of appetite while an eating disorder is any of a range of psychological or physiological disorders characterized by abnormal or distorted eating habits and include illnesses in which people experience severe disturbances in their eating behaviors and related thoughts and emotions.

Anorexia nervosa is an eating disorder characterized decreased body weight of about 85% of normal weight for their age 2.) fear of gaining weight 3.) distorted self-body image and 4) lack of menstruation for greater than 3 months if female (Watson & Bulik, 2013). It should be of great note that anorexia nervosa is not the same thing as anorexia as anorexia nervosa is an eating disorder whereas anorexia is the loss of appetite and thus is entirely different.

Chemically, aripiprazole is 7-[4-[4-(2,3-dichlorophenyl)piperazine-1-yl]butoxy]-3,4-dihydro-1H-quinolin-2-one.

An individual is any animal, most preferably a human.

Agonist is a something that increases activity of a receptor (Kenakin, 1987).

Antagonist is something that competes with an agonist for binding to a receptor, helping to block the action of an agonist on the receptor (Kenakin, 1987).

An inhibitor is something that inhibits the receptor from activating (Kenakin, 1987).

An inverse agonist is something that decreases basal activity of a receptor and can be effective at reducing basal activity of a receptor without the presence of an agonist. It may also be called a negative antagonist (Kenakin, 1987).

A serotonin receptor is defined as a receptor in the body having a corresponding activity related to serotonin or mimics of serotonin and may include any subtypes of serotonin receptors such as but not limited to: 5-HT1A 5-HT1B 5-HT1D 5-HT2A 5-HT2B 5-HT2C 5-HT3, 5-HT5A, 5-HT6, 5-or HT7.

A dopamine receptor is defined as a receptor in the body having a corresponding activity related to dopamine or mimics of dopamine and may include any subtypes of dopamine receptors such as but not limited to: D1, D2, D3, D4, D5.

A compound may be defined as anything that produces an effect on the activity of a receptor or another molecule.

A therapeutically effective amount is an amount of a compound or agent that creates a biological or health response in a cell, tissue, system, animal, or human which includes alleviation, lessening of the symptoms of the disease being treated, or prevent or slows the progress of the disease.

Local means just one individual area, localized to just one area and not an entire body.

Systemic means influencing more than one area usually by entering the blood stream.

Intranasal means lying within or administered by way of the nasal structures or one in which something is insufflated through the nose. This may occur from a device placed inside the nasal passage, around the nasal passage, or even outside of the nasal passage or nose.

MAD stands for Intranasal Mucosal Atomization Device

The present invention is for the use of a nasal route of administration of aripiprazole for treatment of eating disorders, specifically anorexia nervosa (AN). This nasal route of administration may include but is not limited to nasal administration via pump spray bottle, syringe with mucosal atomization device or intranasal mucosal atomization (MAD) device also called nasal atomizer, eye dropper pump bottle spray, nasal administration via pressurized canister, nasal administration via aerosol inhaler, or via a pmdi adapter. It may occur via a pump spray bottle or via a syringe with attached intranasal mucosal atomization (MAD) device. It may also occur via a nasal spray, nasal drops delivering device, nasal douche, or other type of dispenser unit used for the transfer of aripiprazole into the nasal passage or passages for treatment of eating disorders. It may contain a canister or chamber capable of holding the drug and base and whatever is needed to deliver the drug to the intended area. The device may also be porous or nonporous, a diffuser or non-diffuser, metered dosage or nonmetered dosage, sprayer, mister, disperser, deflector, or a device for propelling the molecules, pressurized, or a negative pressure apparatus. This transport device may be made with hydrophobic or hydrophilic material of any density and pressure as well as various types of material such as plastic, metal, glass or carbon based in a form such as but not limited to mesh, filter or screen. The device may be pressured or not, sealed or not, sterilized or not, and in a vacuum or not. The device may also include parts that are porous or nonporous.

In some embodiments, this nasal route of administration of aripiprazole may include compounds or combinations of compounds are part of physiologically acceptable carriers, vehicles, excipients, diluents, solvents, buffers, salts, or other agents. The drug delivery system may include prefilled syringes or cartridges. It may include manual or auto injection as well as dispersions, suspensions, or colloids. Vehicles used in pharmaceutical formulations are mainly liquid bases which carries drugs and other excipients in dissolved or dispersed state or products that help with solubility, stability and bioavailability of active ingredient. This nasal route of administration may also include but is not limited to various liposome coated drug delivery system, intrapleural route of administration, via ionic administration, intraendoplasm, intraectoplasm, intracellularly, extracellularly, or via lipid bilayer membrane administration. The route may also be via a prodrug. In some embodiments, this delivery mechanism may also include specific mucosal adhesives, enhancers, penetrative compounds, constrictors, vasodilators, permeability modifiers, or nanoparticle vehicles, cytokines, immune compound modifiers, and various tissue factors such as brain derived neurotrophic factor (BDNF). The size of the cartridge, syringe, needle, or intranasal mucosal atomization (MAD) device may vary in size and diameter if applicable.

In some embodiments, the compounds or combinations of compounds may be in any form such solid, liquid, or gas, foam, or powder. It may also be condensed, frozen, heated, evaporated, sublimed, melted, vaporized, or deposited. The temperature of the base or device may also vary and the device, base, or compound may be stored refrigerated, frozen, at room temp, or hot and the compound or compounds may be administered hot, at room temp, cold, or frozen. The device may be pressured or not, sealed or not, sterilized or not, and in a vacuum or not. The person may have to breathe or hold their breath when administering the drug. The compound or compounds may also be manufactured in a way that may or may not use misting, dissolving, granulating, levigating, emulsifying, crushing, encapsulating, entrapping, or tableting or through various buffers, dragees, capsules, gels, liquids, syrups, powders, sugar fillers, oil or starch fillers, slurries, suspensions, fillers, or suitable coatings such as gels, organic or solvent mixtures, pigments or substances such as gelatin, plasticizer, starches, oils, paraffin, glycols, push-fit, sustained release capsules, sprays, fine sprayed, mist, misted, diluted, shaken, dissolved, capsules, cartridges, inhalers, bolus injections, stabilizers, dispersing agents, dispersible tables, resins, and or soluble derivatives and phases.

This nasal route of administration may include a unique base for transporting the aripiprazole molecules. This may include one or a combination of any of the following: water, glycerol, sorbitol, benzyl alcohol, alcohol, distilled water, polar or nonpolar solutes, as well as organic and or non-organic solutes and long with any of the medication needed. Base ingredients may also contain both active and inactive ingredients as well as preservatives, sweeteners, buffers, stabilizers, vehicles, solvents, chelating agents, vaporizing agents, boiling point changers, dissolvable agents, dispersible agents, carriers, or vehicles or agents to improve smell of product. Some of these ingredients may include but are not limited to benzalkonium chloride (a preservative), disodium ethylenediaminetetraacetate (a stabilizer), sodium chloride, hydrochloric acid, and purified water or distilled water. It may also include microcrystalline cellulose, carboxymethylcellulose sodium, dextrose, benzalkonium chloride, polysorbate 80, and phenylethyl alcohol or malic acid. In addition, the base may contain propylene glycol, glycerin, alcohol, polyhydric alcohol and water for injection which is used alone or in combination. Preservatives are included in pharmaceutical solutions to control the microbial bioburden of the formulation having broad spectrum of antimicrobial activity, must be chemically and physically stable over the shelf-life of the product and have low toxicity. Preservative can be selected from group but not limited to alcohol, benzyl alcohol, chlorbutol, chlorocresol, alkyl esters of paraben, phenol, phenyl ethanol, sodium benzoate, antimicrobial solvents like propylene glycol, chloroform, alcohol, benzyl alcohol, chlorbutol, chlorocresol, alkyl esters of paraben, phenol, phenyl ethanol, polysorbate, sorbitan laurate, sodium benzoate, sodium phosphate dibasic anhydrous, sodium dihydrogen phosphate monobasic dihydrate, antimicrobial solvents like propylene glycol, chloroform, and water. Sweetener that could be selected from but not limited to are sucrose, liquid glucose, glycerol, sorbitol, saccharin sodium and aspartame to impart sweetness to the formulation. Chelating agent is used for drug stabilization, to maintain potency of active ingredients and to stabilize colors and flavors. Chelating agents that may be selected from but not limited to are citric acid monohydrate, disodium edetate, dipotassium edetate, edetic acid, fumaric acid, malic acid, phosphoric acid, sodium edetate, tartaric acid and trisodium edetate, polyurethane glycol, propylene glycol, butylate hydroxtolune, and saccharin.

This drug administration may include local or systemic effects. However, systemic administration and systemic effects are most likely necessary for the medication to be therapeutically effective. This route of administration includes transfer of aripiprazole to the olfactory system and then the central nervous system. This may be delivered to and act on any of the following areas: outer and intranasal cavity (both upper and lower nasal cavity), olfactory mucosa, olfactory epithelium, olfactory cells, cribriform plate, cranial cavity, blood brain barrier, brain tissue and cerebrospinal fluid, intracellular, extracellular, cytoplasm, central nervous system or peripheral nervous system. It may also act on or be transported to around or through the blood brain barrier, brain tissue and cerebrospinal fluid, intracellular, extracellular, cytoplasm, cellular or molecular transport machinery of the body, vesicles, nanoparticles, microparticles, central or peripheral nervous system, olfactory receptor neurons, basal cells, supporting cells, astrocytes, sensory neurons, motor neurons, pyramidal neurons, betz cells, or microglia as well as various types of neurons such as but not limited to unipolar neurons, bipolar neurons, psuedounipolar neurons, or multipolar neurons. For example, one way this transport may occur would be a route of administration where aripiprazole molecules are sprayed into the nasal cavity and olfactory transfer then occurs across the single epithelial cell layer to blood vessels via either transport to the olfactory nerve cells and the olfactory bulb or through the perineural space by the olfactory nerve cells to the cerebrospinal fluid neighboring the olfactory bulbs and then subsequently reach the brain and central nervous system.

This route of administration may be delivered primarily through the nasal cavity intranasally but may also have its effect in part via delivery of the compound or compounds to the lungs, skin, muscle, or conjunctiva. As such, it is possible that this nasal route of administration may in part be transported into the lungs during inhalation such as via an inhaler and therefore may play a systemic effect via absorption of blood vessels in the lungs. This route of administration may also include the transfer of aripiprazole passively or actively (such as via diffusion or via cellular or molecular transport) as well as naturally or artificially (such as via bodily forces or via physical forces such as pressure, electrically, magnetically, or mechanically driven into the nasal cavity or central nervous system). It may also include the transfer of the drug to the brain from the blood circulation through the blood brain barrier via passive diffusion or may bypass it and enter the brain directly through such methods such as the olfactory epithelium in the nasal cavity.

This route of administration may be used for pediatric or adult individuals. This novel route of nasal administration of aripiprazole may be used for treatment of any eating disorder including to but not limited to anorexia nervosa, atypical anorexia nervosa, bulimia nervosa, binge-eating disorder, purging disorder, or any combination of multiple types of eating disorders, as well as certain syndromes such as night eating syndrome, carcinoid syndrome, or serotonin syndrome or any diseases or symptoms comorbid with an eating disorder such as anxiety, depression, or body dysmorphia in those with an eating disorder. Moreover, this nasal route of administration may be used for treating various aspects of the eating disorder such as biological, psychological, psychiatric, medical, or social problems or help treat any symptoms associated with or comorbid with an eating disorder.

The dose of the compound or combination of compounds may be anything that is a therapeutically effective amount. In some embodiments, the dosage of aripiprazole or any compound or combination of the compounds described herein this invention can be more or less than the standard dose given for other diseases or disorders and may be less than the maximum dose allowed and more or less than the efficacious dose when the compound is administered alone. Examples of various dosages may be but are not limited to 0.01 mg, 0.1 mg, 1 mg, 5 mg, 10 mg, 20 mg, and 30 mg, 60 mg, 120 mg, 240 mg. The dose may also be less than standard doses used for oral administration such as 1/1000 of the standard dose (0.0001 mg) up to 1000× the standard dose (30000 mg) but will most likely be anywhere between 0.1 mg to 30 mg.

The specific combination, exact route of administration, and dosage for the compounds or combinations of compounds of the invention may be chosen by a medical professional and the doses can vary in amounts based on age, individual, gender, sex, condition, situation, or exact route of administration and dose interval and amount may be adjusted depending on the situation.

In some embodiments, the compound or combination of compounds can be administered sequentially or at varying times and the compounds or combination of compounds can be administered using the same route or different routes. It may be administered quickly or slowly before or after other treatments and may include rapid release, extended release, or sustained released dosages The compounds may be encapsulated in the same container such as a pill or tablet or may be administered in separate containers and taken at the same time or at separate times. This combination may or may not be occurring at temporally similar times. One can be given first, then the other. Or one may be given first and then wait for the effects to be achieved before the other is given or vice versa. Or one compound may be given first and then eliminated before the second compound or combination of compounds is given or vice versa or they may be given at the same time in differing absorption rates or routes. This route may be beneficial and administered due to differences in rates of metabolism, absorption, onset of action (such as faster or slower), half-life, toxicity, reliability, or elimination and excretion compared to other routes. For example, nasal route of administration may produce effects faster than oral route of administration. The nasal route of administration may also help increase treatment acceptance and compliance via improving areas the drug can be administered such as at home instead of solely at a hospital such as if given intravenously instead.

It may include local or systemic effects. However, systemic administration is most likely needed as the medication probably needs to reach the brain to be therapeutically effective . One possible example includes the transfer of the drug molecules across the single epithelial cell layer directly to the systemic blood circulation without first-pass hepatic and intestinal metabolism to the brain and into the neurons or synapses to create an effect. It may also include administration into the lining of the nasal cavity and include absorption via the mucosal linings into blood vessels and brain or may also but not necessarily include administration into the respiratory tract and thus administration into the lungs and subsequent blood vessels and brain.

In addition, the route of administration may include aripiprazole used as a serotonin antagonist or a dopamine agonist or as part of a combination of the two for treatment of an eating disorder. This may be Abilify or a generic brand of aripiprazole. Moreover, this nasal route of administration of aripiprazole may act systemically or locally in a way that either directly or indirectly reduces serotonin or alters serotonin receptor binding such as an antagonist, inhibitor, inverse agonist, or partial agonist on any of the following serotonin receptors: 5-HT1A 5-HT1B 5-HT1D 5-HT2A 5-HT2B 5-HT2C 5-HT3, 5-HT5A, 5- HT6, 5-or HT7 for treatment of any eating disorder including to but not limited to anorexia nervosa, atypical anorexia nervosa, bulimia nervosa, binge-eating disorder, purging disorder, or any combination of multiple types of eating disorders, as well as any syndrome disease such as night eating syndrome, carcinoid syndrome, or serotonin syndrome or any diseases or symptoms comorbid with an eating disorder. This compound may also alter the serotonin receptor transporter and may or may not also alter dopamine levels or dopamine receptor binding.

Additionally, this nasal route of administration of aripiprazole may act systemically or locally in a way that either directly or indirectly increases dopamine or alters the dopamine receptor binding specifically a partial or full agonist of the dopamine receptors D1, D2, D3, D4, D5 receptors along with any alteration in the dopamine transporter or dopamine active transporters that may or may not also alter serotonin levels or serotonin receptor binding for treatment of any eating disorder including to but not limited to anorexia nervosa, atypical anorexia nervosa, bulimia nervosa, binge-eating disorder, purging disorder, or any combination of multiple types of eating disorders, as well as any syndrome disease such as night eating syndrome, carcinoid syndrome, or serotonin syndrome or any diseases or symptoms comorbid with an eating disorder.

Moreover, this nasal route of administration of aripiprazole may act systemically or locally in a way that directly or indirectly acts as a combination of a serotonin antagonist and a dopamine agonist for treatment of an eating disorder. This nasal route of administration of aripiprazole could be a used either by itself or in combination with a compound or compounds that could lead to an effect that both reduces serotonin or alters serotonin receptor binding or its active transporters such as an antagonist, inhibitor, inverse agonist, or partial agonist on any of the following serotonin receptors: 5-HT1A 5-HT1B 5-HT1D 5-HT2A 5-HT2B 5-HT2C 5-HT3, 5-HT5A, 5-HT6, 5-or HT7 as well as increases dopamine or alters dopamine receptor binding specifically a partial or full agonist of the dopamine receptors D1, D2, D3, D4, D5 receptors along with any alteration in the dopamine transporter or dopamine active transporters that may or may not also alter serotonin levels or serotonin receptor binding for treatment of any eating disorder including to but not limited to anorexia nervosa, atypical anorexia nervosa, bulimia nervosa, binge-eating disorder, purging disorder, or any combination of multiple types of eating disorders, as well as a syndrome such as night eating syndrome, carcinoid syndrome, or serotonin syndrome or any diseases or symptoms comorbid with an eating disorder.

This novel compound or combination of compounds used for treatment of anorexia may or may not also be possibly taken in combination with foods or other types of pharmaceuticals such as anxiety medications, depression medications, SSRIs, SNRI, MAO-I, TCA, antipsychotic medication, anti-manic medication, anti-phobia medication or PTSD medications. Examples include but are not limited to acrivastine, alprazolam, amisulpride, amitriptyline, amitryptilinc, amoxapinc, bripiprazole, clomipraminc, antipsychotics, astemizole, azatadine, benzodiazepines, brompheniramine, bupropion, carbinoxamine, cetirizine, chlordiazepoxide, chlorpheniramine, clemastine, cyproheptadine, D-cycloserine, desipramine, desipramine, desloratadine, dimenhydrinate, diphenhydramine, divalproex, doxepin, doxepin, doxylamine, duloxetine, duloxetine, escitalopram, escitalopram, escitaloram, Etrafon, fexofenadine, fluoxetine, flurazepam, fluvoxamine, Haldol, haloperidol, hydroxyzine, imipramine, inapsine, isocarboxazid, ktotifen, leocabastine, lithium, lithium carbonate, lithium citrate, loratdine lorazepam, manserin, maprotiline, maprotiline, meclizine, mellaril, mepramine, mirtazapine, moban, mood stabilizers, navane, paroxetine, paroxetine, pentobarbital, permitil, phenelzine, phenelzine, phenergen, prochlorperazine, prolixin, promethazine, protryptiline, quazepam, reglan, Risperdal, secobarbital, sertraline, sibutramine, stelazine, taractan, temazepam, tipelennamine, thiridazine trazodone, triavil, triazolam, trilafon, trimipramine, triprolidine, valium, valproate, venfalazine, venlafaxine, venlafaxine, zaleplon, ziprasidone, zolpidem, bifeprunox, N-desmethylclozapine, clozapine, SL-88-0338, olanzapine, quetiapine, preclamol (3-PPP), SLV-313 SR, SSR 181507, WAY-100,635, WAY-100,135, p-MPPI, MM-77, pindobind 5-hydroxytryptamine1A, sumatriptan, almotriptan, naratriptan, frovatriptan, rizatriptan, zomitriptan, cabergoline, amantadine, lisuride, pergolide, ropinirole, pramipexole, bromocriptine, and histamine antagonists.

This nasal route of administration may be used for the treatment of an eating disorder such as but not limited to anorexia nervosa, atypical anorexia nervosa, bulimia nervosa, binge-eating disorder, purging disorder, or any combination of various eating disorders as well as any syndrome disease such as night eating syndrome, carcinoid syndrome, or serotonin syndrome or any diseases or symptoms comorbid with an eating disorder. These symptoms comorbid with an eating disorder may include but are not limited to: alpha adrenergic side effect, histaminic side effect, anticholinergic side effect, parkinsoniam movement disorder, movement disorder dyskinesia, dystonia, gastrointestinal problems, enlarged abdomen, diabetes, abnormal glucose, lipid, triglyceride, cholesterol, or creatine phosphokinase levels, flushing, nausea, vomiting, abdominal pain, diarrhea, liver failure, pulmonary problems, stroke, cerebrovascular events, cardiovascular disease, heart problems, AV block, angina pectoris, premature atrial contractions, T wave inversion, ventricular extrasystoles, ST depression, myocarditis, abnormal accommodation, hypotension, hypertension, tremors, increased risk of death, nerve pain, muscle pain, neurological problems, amnesia, anxiety, depression, post-traumatic stress disorder, obsessive compulsive disorder, bipolar disorder, schizophrenia, paranoia, psychosis, suicidality, catatonic reaction sedation, emotional liability, nervousness, apathy, abnormal body temperature, fever, infection, acne, diminished sexual desire, delirium, migraine, menstruation irregularities, dementia, Alzheimer's disease, memory loss, personality disorders, social anxiety disorder, ADHD, or medication tolerance.

### REFERENCES

Audenaert, K., et al., Decreased 5-HT2a receptor binding in patients with anorexia nervosa. Journal of Nuclear Medicine, 2003. 44(2): 163-169.
Barrett, L. F., et al., The amygdala and the experience of affect. Social Cognitive and Affective Neuroscience, 2007. 2(2): 73-83.
Bergen, A. W., et al., Candidate genes for anorexia nervosa in the 1p33-36 linkage region: serotonin 1D and delta opioid receptor loci exhibit significant association to anorexia nervosa. Mol Psychiatry, 2003. 8(4): 397-406.
Birmingham, C. L., et al., The mortality rate from anorexia nervosa. Int J Eat Disord, 2005. 38(2): 143-6.
Bissada H, Tasca G A, Barber AM, Bradwejn J: Olazapine in the treatment of low body weight and obsessive thinking in women with anorexia nervosa: a randomized, double-blind, placebo-controlled trial. Am J Psychiatry 2008; 165: 1281-1288
Blonder, L. X. and J. T. Slevin, Emotional dysfunction in Parkinson's disease. Behavioural Neurology, 2011. 24(3): 201-217
Bloss, C. S., et al., Genetic Association of Recovery from Eating Disorders: The Role of GABA Receptor SNPs. Neuropsychopharmacology, 2011. 36(11): 2222-2232.
Bocchio, M., McHugh, S. B., Bannerman, D. M., Sharp, T., & Capogna, M. (2016). Serotonin, Amygdala and Fear: Assembling the Puzzle. Frontiers in Neural Circuits, 10. doi:ARTN 24 10.3389/fncir.2016.00024
Ellinson, Z., et al., Functional anatomy of calorie fear in anorexia nervosa. Lancet, 1998. 352(9135): 1192-1192.
Engel, S. G., et al., The Role of Affect in the Maintenance of Anorexia Nervosa: Evidence From a Naturalistic Assessment of Momentary Behaviors and Emotion. Journal of Abnormal Psychology, 2013. 122(3): 709-719.
Ferguson, C. P., La Via, M. C., Crossan, P. J., & Kaye, W. H. (1999). Are serotonin selective reuptake inhibitors effective in underweight anorexia nervosa? Int J Eat Disord, 25(1), 11-17. Frank, G. K., Could dopamine agonists aid in drug development for anorexia nervosa? Front Nutr, 2014. 1: 19.
Frank, G. K., et al., Reduced 5-HT2A receptor binding after recovery from anorexia nervosa. Biological Psychiatry, 2002. 52(9): 896-906.
Heaner, M. K., & Walsh, B. T. (2013). A history of the identification of the characteristic eating disturbances of Bulimia Nervosa, Binge Eating Disorder and Anorexia Nervosa. Appetite, 71: 445-448.
Frisch, A., Laufer, N., Danziger, Y., Michaelovsky, E., Leor, S., Carel, C., . . . Weizman, A. (2001). Association of anorexia nervosa with the high activity allele of the COMT gene: a family-based study in Israeli patients. Molecular Psychiatry, 6(2): 243-245. doi:DOI 10.1038/sj.mp.4000830
Frodl, T., et al., Enlargement of the amygdala in patients with a first episode of major depression. Biol Psychiatry, 2002. 51(9): 708-14.
Galimberti, E., et al., Executive functioning in anorexia nervosa patients and their unaffected relatives. Psychiatry Research, 2013. 208(3): 238-244.
Halmi, K. A., et al., An examination of early childhood perfectionism across anorexia nervosa subtypes. International Journal of Eating Disorders, 2012. 45(6): 800-807.
Halmi K. The perplexities of conducting randomized double-blind placebo-controlled treatment trials in anorexia nervosa patients. Am J Psychiatry, 2008. 165(10): 1281-8.
Halmi K A, Agras W S, Crow S, Mitchell J, Wilson G T, Bryson S W, Kraemer H C: Predictors of treatment acceptance and completion in anorexia nervosa: implications for future study designs. Arch Gen Psychiatry 2005; 62: 776-781
Joos, A. A. B., et al., Amygdala hyperreactivity in restrictive anorexia nervosa. Psychiatry Research-Neuroimaging, 2011. 191(3): 189-195.
Kaye, W. H., et al., Serotonin alterations, in anorexia and bulimia nervosa: New insights from imaging studies. Physiology & Behavior, 2005. 85(1): 73-81.
Kaye, W. H., et al., Altered Serotonin Activity in Anorexia-Nervosa after Long-Term Weight Restoration—Does Elevated Cerebrospinal-Fluid 5-Hydroxyindoleacetic Acid Level Correlate with Rigid and Obsessive Behavior. Archives of General Psychiatry, 1991. 48(6): 556-562.
Kenakin, T. (1987). Agonists, partial agonists, antagonists, inverse agonists and agonist/antagonists? Trends in Pharmacological Sciences, 8(11): 423-426.
Kienast T, Hariri A, Schlagenhauf F, Wrase J, Sterzer P, Buchholz H, Smolka M, Grunder G, Cumming P, Kumakura Y, Bartenstein P, Dolan R, and Heinz A. Dopamine in amygdala gates limbic processing of aversive stimuli in humans. Nature Neuroscience, 2008; 11(12): 1381-2. Lebow, J., Sim, L. A., Erwin, P. J., & Murad, M. H. (2013). The effect of atypical antipsychotic medications in individuals with anorexia nervosa: a systematic review and meta-analysis. Int J Eat Disord, 46(4), 332-339. doi:10.1002/eat.22059
Lin J, Arthurs J, and Reilly S. (2017). Conditioned taste aversions: From poisons to pain to drugs of abuse. Psychon Bull Rev. 2017 Apr;24(2): 335-351.
Malczyk Z and Oswiecimska J. Gastrointestinal complications and refeeding guidelines in patients with anorexia nervosa. Psychiatr. Pol. 2017; 51(2): 219-229.
Oades, R. D. and G. M. Halliday, Ventral tegmental (A10) system: neurobiology. 1. Anatomy and connectivity. Brain Res, 1987. 434(2): 117-65.
Oldershaw, A., et al., Emotion generation and regulation in anorexia nervosa: A systematic review and meta-analysis of self-report data. Clinical Psychology Review, 2015. 39: 83-95.
Oldershaw, A., et al., Emotional Processing Following Recovery from Anorexia Nervosa. European Eating Disorders Review, 2012. 20(6): 502-509.
Pessoa, L., & Adolphs, R. (2010). Emotion processing and the amygdala: from a ‘low road’ to ‘many roads’ of evaluating biological significance. Nature Reviews Neuroscience, 11(11): 773-782. doi:10.1038/nrn2920
Phillipou, A., S. L. Rossell, and D. J. Castle, The neurobiology of anorexia nervosa: a systematic review. Aust N Z J Psychiatry, 2014. 48(2): 128-52.
Silvers, J. A., McRae, K., Gabrieli, J. D. E., Gross, J. J., Remy, K. A., & Ochsner, K. N. (2012). Age-Related Differences in Emotional Reactivity, Regulation, and Rejection Sensitivity in Adolescence. Emotion, 12(6): 1235-1247. doi:10.1037/a0028297
Vocks, S., et al., Effects of gustatory stimulation on brain activity during hunger and satiety in females with restricting-type anorexia nervosa: An fMRI study. Journal of Psychiatric Research, 2011. 45(3): 395-403.
Watson, H. J., & Bulik, C. M. (2013). Update on the treatment of anorexia nervosa: review of clinical trials, practice guidelines and emerging interventions. Psychol Med, 43(12): 2477-2500. doi:10.1017/S0033291712002620.
Wild, B., et al., Predictors of outcomes in outpatients with anorexia nervosa—Results from the ANTOP study. Psychiatry Research, 2016. 244: 45-50.
Williams, S. M. and P. S. Goldman-Rakic, Widespread origin of the primate mesofrontal dopamine system. Cereb Cortex, 1998. 8(4): 321-45.
Rosen E, Bakshi N, Watters A, Rosen H, and Mehler P. Hepatic complications of anorexia nervosa. Dig Dis Sci. 2017 Nov;62(11): 2977-2981.

1. The present invention is for the novel use of a nasal route of administration of aripiprazole for the treatment of an eating disorder. This nasal route of administration may include the use of aripiprazole administered via a syringe with attached intranasal mucosal atomization (MAD) device, a pump spray bottle, a nasal inhaler, or other type of nasal dispenser unit. This novel nasal route of administration of aripiprazole will primarily be used for anorexia nervosa but may also include other eating disorders such as but not limited to atypical anorexia nervosa, bulimia nervosa, binge-eating disorder, purging disorder, or any combination of multiple types of eating disorders. This nasal route of administration of aripiprazole may help by acting as a partial serotonin antagonist and or a partial dopamine agonist. This nasal route of administration or aripiprazole may also be important for correcting the neurotransmitter imbalance seen in those with an eating disorder and improving symptoms such as weight abnormalities, anxiety, depression, or other problems associated with an eating disorder. Moreover, this particular route of administration may help with reducing treatment refusal and improving treatment compliance by reducing anxiety from oral phobia, reduce complications from other route of administrations such as from refeeding syndrome, liver problems, or physical pain from other routes of administration, and finally could help through different absorption and metabolism rates compared to other routes of administration. One example this route could occur is aripiprazole molecules are sprayed into the nasal cavity where they are transferred via the olfactory space across the single epithelial cell layer to blood vessels via transport to the olfactory nerve cells or the olfactory bulb or through the perineural space by olfactory nerve cells to the cerebrospinal fluid neighboring the olfactory bulbs and then subsequently reach the brain and central nervous system where they may produce local and or systemic effects in order to treat the eating disorder.
